(19) United States
(12) Patent Application Publication
(10) Pub. No.: US 2007/0224131 A1
(43) Pub. Date: Sep. 27, 2007

(54) Pet chew article

(76) Inventors: James E. McCollum, St. Paul, MN (US); Edward J. Goettert, Dellwood, MN (US); Li Nie, Parkville, MO (US); Sukh D. Bassi, Atchison, KS (US); Michael D. Parker, Lawrence, KS (US); Norman Worthy, Atchison, KS (US); Clodualdo C. Maningat, Platte City, MO (US)

Correspondence Address: 3M INNOVATIVE PROPERTIES COMPANY, PO BOX 33427, ST. PAUL, MN, 55133-3427, US

(21) Appl. No.: 11/385,373

(22) Filed: Mar. 21, 2006

(51) Int. Cl. A61K 47/00; A61K 31/202

(52) U.S. Cl. 424/49; 424/442

(57) ABSTRACT

Pet chew articles comprising a consumable body having a shape defined by an exterior surface and comprising a main portion and at least one protrusion extending therefrom with the proviso that the shape of the body is such that no point in the interior of the body is more than about 0.2 inch (⅕ inch) from the portion of the exterior surface nearest to the point. As a result, any pieces of the article that are ingested by a consuming animal are small enough that they will not become lodged within the animal's digestive tract. Also a method for improving the dental health of animals comprising chewing or eating by an animal of such an article.

### FIELD

The invention relates to edible chew articles that improve the dental health of animals such as pets. The invention also relates to a process for preventing tartar formation on and tartar removal from animal's teeth by chewing on such chew articles.

### BACKGROUND

Dental calculus, or tartar as it is sometimes called, is a deposit which forms on the surfaces of the teeth at the gingival margin. Supragingival calculus appears principally in the areas near the orifices of the salivary ducts; e.g., on the lingual surfaces of the lower anterior teeth and on the buccal surfaces of the upper first and second molars, and on the distal surfaces of the posterior molars. Calculus develops from a biofilm precursor through calcification. This biofilm is known as plaque. Mature calculus consists of an inorganic portion which is largely calcium phosphate arranged in a hydroxylapatite crystal lattice structure similar to bone, enamel and dentine. An organic portion is also present and consists of desquamated epithelial cells, leukocytes, salivary sediment, food debris and various types of microorganisms. As the mature calculus develops, it becomes visibly white or yellowish in color unless stained or discolored by some extraneous agency. In addition to being unsightly and undesirable from an aesthetic standpoint, the mature calculus deposits are constant sources of irritation of the gingiva and thereby are a contributing factor to gingivitis and other diseases of the supporting structures of the teeth, the irritation decreasing the resistance of tissues to endogeneous and exogenous organisms.

The microorganisms that the plaque and calculus harbor not only can cause infections in the oral cavity, but also cause breath malodor due to metabolism of sulfur containing proteinaceous materials from the desquamated epithelial cells, food debris, etc. Breath malodor is a common complaint of owners of companion animals and a variety of methods have been developed to absorb or mask this odor.

A wide variety of chemical and biological agents have also been suggested in the art to retard calculus formation or to remove calculus after it is formed in humans and in animals, particularly pets. Mechanical removal of this material is done routinely in humans but is more problematic with regard to animals.

Illustrative chewing articles for pets are disclosed in, for example, German Patent No. 3,426,203 (Hans), U.S. Pat. No. 3,882,257 (Cagle), U.S. Pat. No. 4,145,447 (Fisher et al.), U.S. Pat. No. 5,000,943 (Scaglione et al.), U.S. Pat. No. 5,296,209 (Simone et al.), and U.S. Pat. No. 5,618,518 (Stookey). U.S. Pat. Nos. 3,701,830 (Weinrich et al.), U.S. Pat. No. 4,364,925 (Fisher), U.S. Pat. No. 3,194,738 (Harrison et al.), and U.S. Pat. No. 3,686,393 (Woodruff et al.) disclose the use of enzymes for inhibiting plaque.

A common manner of mechanical removal of plaque and calculus is to provide the animals with chew articles. The chewing and gnawing action provides abrasive and mechanical action against the deposits of plaque and calculus on the teeth, resulting in their diminution and removal. A common form of chew articles are made of edible materials which break into pieces and are swallowed as the animal chews on them. A concern with some such chew articles is that they break into relatively large pieces which can be swallowed but which do not pass easily through the animal's digestive system. In some instances animals have needed medical attention to assist with passage or removal of ingested pieces of chew articles; in some instances animals have suffered injury or even died because of ingested pieces of chew articles have become lodged in their digestive tracts.

A need exists for pet chew articles that provide effective dental benefits while not resulting in dangerously large pieces that can be ingested and become lodged within the animal's digestive tract.

### SUMMARY

The present invention provides chew articles that promote dental health of the consuming animal and a process for inhibiting tartar formation on and facilitating tartar removal from the animal's teeth by chewing on such chew articles. The invention provides an easy, effective way for pet owners to maintain the oral health of their pet animals, e.g., typically cats and dogs.

In brief summary, a pet chew article of the invention comprises a consumable body having a shape defined by an exterior surface and comprising a main portion and at least one protrusion extending therefrom with the proviso that the shape of the body is such that no point in the interior of the body is more than about 0.2 inch (⅕ inch) (5.1 millimeter) from the portion of the exterior surface nearest to the point. As a result of this size and shape limitation, any pieces of the article that are ingested by a consuming animal are small enough that they will not become lodged within the animal's digestive tract.

Briefly summarizing, the process of the invention comprises chewing or eating a pet chew article of the invention by an animal.

In accordance with the present invention, an animal such as a pet will undergo a decolonizing of at least a portion of the oral cavity of a subject of microorganisms. As a result, the dental health of the animal will be safely improved.

### BRIEF DESCRIPTION OF DRAWING

The invention will be explained with reference to the drawing wherein:

FIG. 1 is an isometric or perspective view of an illustrative embodiment of the invention;

FIG. 2 is a top view of an other illustrative embodiment of the invention;

FIG. 3A is a schematic cross-sectional view of the embodiment shown in FIG. 1;

FIG. 3 is a schematic cross-sectional view of another embodiment of the invention;

FIG. 4 is a top view of another illustrative embodiment of the invention;

FIG. 5 is a top view of still another illustrative embodiment of the invention;

FIG. 6 is a top view of still another illustrative embodiment of the invention;

FIG. 7 is a schematic cross-sectional view of the embodiment shown in FIG. 5;

FIG. 8 is a top view of still another illustrative embodiment of the invention; and

FIG. 9 is a schematic cross-sectional view of the embodiment shown in FIG. 7.

The figures, which are idealized and not to scale, are intended to be merely illustrative and non-limiting.

### DETAILED DESCRIPTION OF ILLUSTRATIVE EMBODIMENTS

Configuration

As described above, a pet chew article of the invention comprises a molded consumable body having a shape defined by an exterior surface and comprising a main portion and at least one protrusion extending therefrom with the proviso that the shape of the body is such that no point in the interior of the body is more than about 0.20 inch (⅕ inch) (5.1 millimeters), preferably no more than about 0.1875 inch ( 3/16 inch) (4.8 millimeters), more preferably no more than about 0.15 inch (3.8 millimeters), and most preferably no more than about 0.125 inch (⅛ inch) (3.18 millimeters), from the portion of the exterior surface nearest to the point. It has been observed, that typically the action of digestive fluids in an animal's digestive tract will penetrate at least 0.1875 inch ( 3/16 inch) (4.8 millimeters) through a piece of typical pet chew material within 24 hours. The penetrated portion of the pet chew material is softened. As a result, any pieces of pet chew articles of the invention that are ingested by a consuming animal are small enough that they will not become lodged within the animal's digestive tract.

As used herein, “chewable” is meant to refer to a material that will break up into pieces when subjected to chewing action. As used herein, “consumable” refers to a material that can be ingested by an animal.

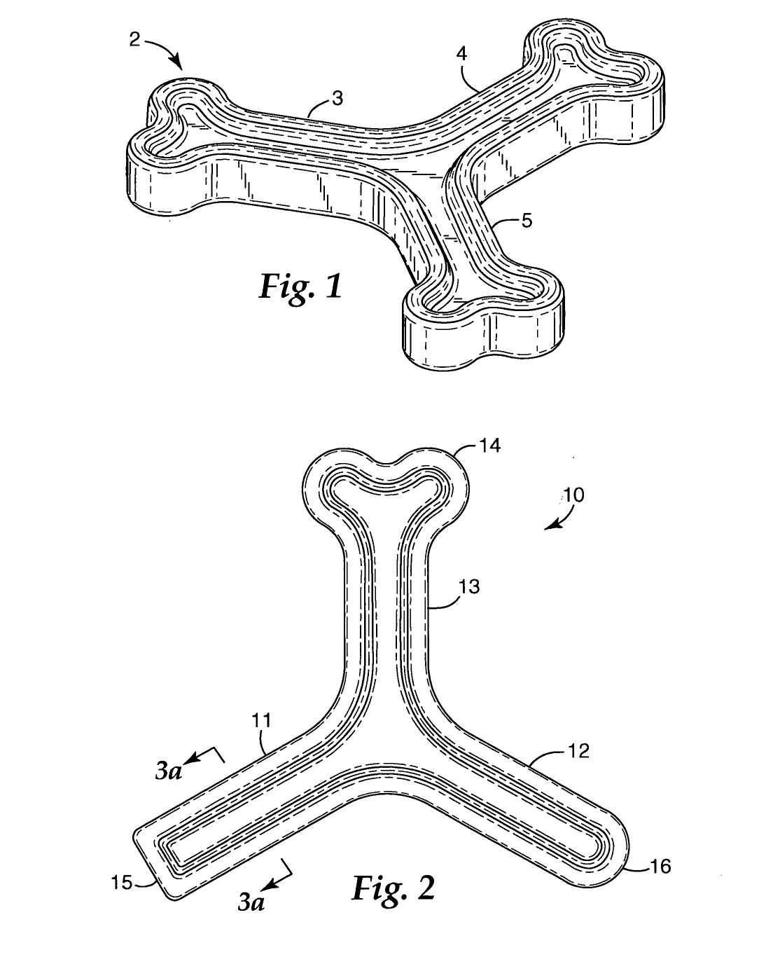

An illustrative embodiment of a pet chew article 2 of the invention is shown in FIG. 1. In this embodiment, article 2 has a generally flat configuration with three extension 3, 4, 5.

An other illustrative embodiment of a pet chew article 10 of the invention is shown in FIG. 2. In this embodiment, article 10 has a generally flat configuration with three extensions 11, 12, 13. The extensions have shaped ends as desired, including for example, bone shaped end 14, square end 15, or round end 16. If desired, the ends may be of other shapes. In some embodiments, all of the extension ends of a particular article will be of similar shape, e.g., shown in FIG. 1.

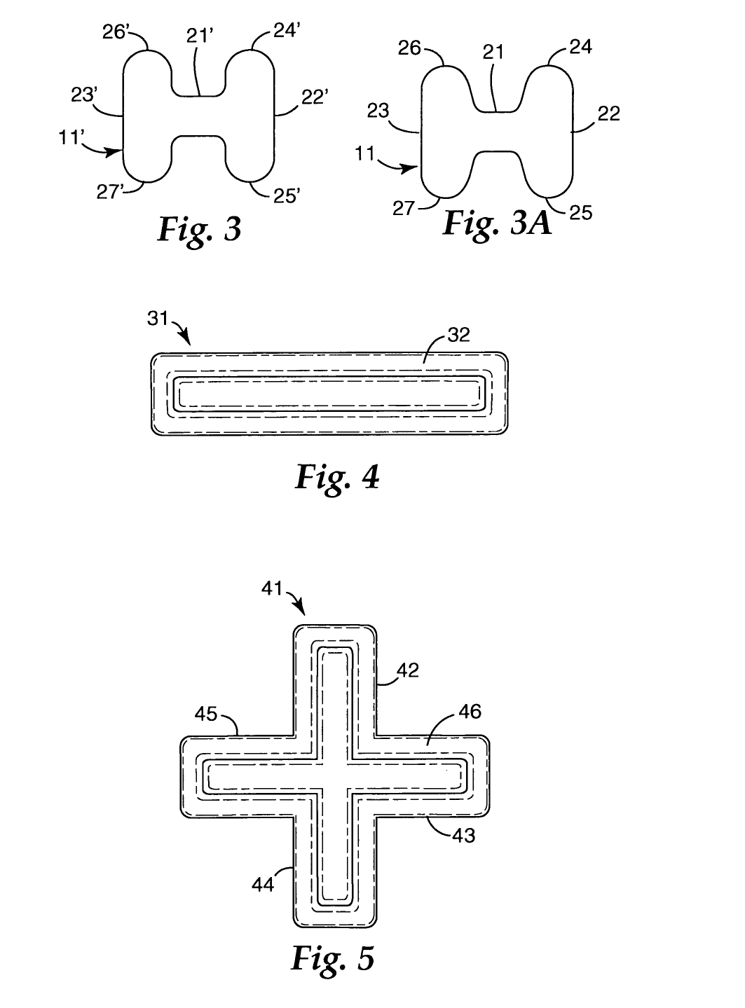

As shown in FIG. 3A, extension 11 has a cross section which is thinner in an interior portion 21 and thicker at the perimeters 22, 23 where it has protrusions 24, 25 extending from the main body and protrusion 26, 27 extending from main body. The extension 11 preferably has protrusions at both edges, i.e., perimeters 22, 23, but in some embodiments will have only one protrusion. This embodiment is a so-called “I-beam” configuration that imports increased dimensional strength to the chew article as well as a shaped surface which provides effective cleaning action when chewed.

FIG. 3 shows a cross-section of pet chew article 11′ having an alternate embodiment wherein the protrusions extend substantially perpendicularly from the main body. It will typically be more preferred for the protrusions to define a slightly oblique angle, e.g., preferably, about 5 to 8 degrees greater than 90° to facilitate removing the article from molding equipment.

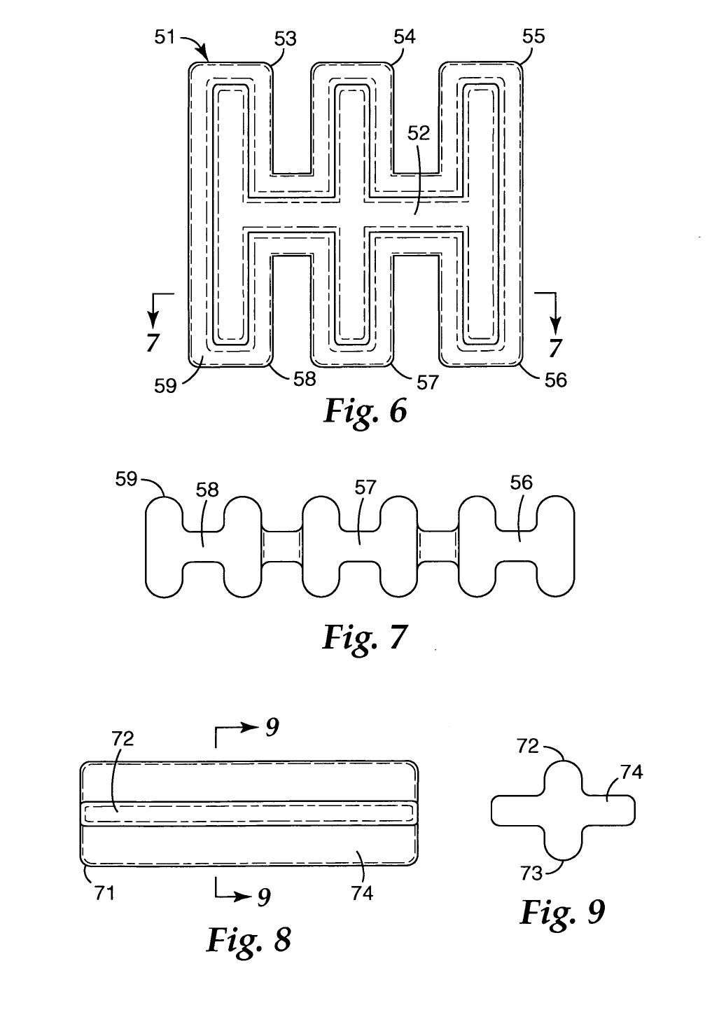

Pet chew articles of the invention can be made in a variety of shapes as desired. FIG. 4 shows an illustrative embodiment wherein article 31 has a generally rectangular shape with protrusion 32 at the perimeter. FIG. 5 shows an illustrative embodiment wherein article 41 has four extensions 42, 43, 44, 45 each with protrusion 46 at the perimeter. FIG. 6 shows an illustrative embodiment wherein article 51 has a central portion 52 from which there are six extensions 53, 54, 55, 56, 57, 58 each with a protrusion 59 at the perimeter. FIG. 7 shows a cross section of article 51. FIG. 8 shows an illustrative embodiment wherein article 71 which is shown in cross section in FIG. 9. Article 71 has a generally rectangular shape with protrusions 72, 73 extending from opposing sides of the main portion 74.

If oriented on an X and Y axis, pet chew articles of the invention will typically be at least about 1 inch (25 millimeters), preferably at least 2 inches (50 millimeters) or more, in their X dimension and typically be at least about 1 inch (25 millimeters), preferably at least 2 inches (50 millimeters) or more in their Y dimensions. In accordance with the invention, they are typically less than 0.4 inch (10 millimeters), preferably less than about 0.375 inch (9.5 millimeters), more preferably less than about 0.3 inch (7.6 millimeters), thick in the Z axis. As used herein, X, Y, and Z axes refer to three mutually perpendicular axes.

Chew Base

The chew base may be made of any of a variety of chewable consumable materials. Illustrative examples include rawhides, biscuits of a variety of compositions, etc.

The chew base should be safe for pets to chew and swallow. Preferably it is of appropriate strength, texture, and firmness to be desirable as a pet chew toy.

The characteristics of the chew base typically define much of the physical properties of the chew article, including substantially defining the density, frangibility, stiffness, and strength of the chew article. Accordingly, it is typically desired to select a chew base that is suitable for forming desired shapes for the chew article in accordance with the invention.

A preferred chew base is so-called modified wheat gluten. Articles made with such chew base can be made with desirable hardness and do not get “gummy” or “slimy” when wet. In addition, such material can be readily molded into desired shape. A second suitable chew base material is gelatinized starch although such materials may not provide chew articles having desired hardness and toughness to achieve optimal levels of dental cleaning. Illustrative examples of suitable chew base compositions are disclosed in U.S. Pat. Nos. 5,747,648, 5,965,708, and 5,977,312 (all Bassi et al.), and U.S. Patent Application Publications 2004/0086616, 2004/0197455, 2005/0008759, and 2005/0214349 (all Nie et al.). As disclosed in those documents, starch and gluten products can be made with very desirable strength. Moreover, such products can be readily controlled in terms of color and opacity.

The chew base is preferably a tough, pliant material that will provide the animal using it with vigorous mechanical action against the teeth as well as entertainment value.

In some embodiments, the chew base may comprise other ingredients to impart improved performance. The other components of the article are blended into the chew base, e.g., by mixing, typically before molding or otherwise shaping the article in accordance with the invention.

For example, an effective amount of an antimicrobial lipid component, e.g., a fatty acid ester of a polyhydric alcohol, a fatty ether of a polyhydric alcohol, alkoxylated derivatives thereof (of either the ester or ether), or combinations thereof, may be incorporated to impart improved antimicrobial activity. In one embodiment, the lipid component includes a C₇ to C₁₄ saturated fatty acid ester of a polyhydric alcohol, a C₈ to C₂₂ unsaturated fatty acid ester of a polyhydric alcohol, a C₇ to C₁₄ saturated fatty ether of a polyhydric alcohol, a C₈ to C₂₂ unsaturated fatty ether of a polyhydric alcohol, an alkoxylated derivative thereof, or combinations thereof, wherein the alkoxylated derivative has less than 5 moles of alkoxide per mole of polyhydric alcohol; with the proviso that for polyhydric alcohols other than sucrose, the esters include monoesters and the ethers include monoethers, and for sucrose the esters include monoesters, diesters, or combinations thereof, and the ethers include monoethers, diethers, or combinations thereof. Preferably, the antimicrobial lipid component includes glycerol monolaurate, glycerol monocaprate, glycerol monocaprylate, propylene glycol monolaurate, propylene glycol monocaprate, propylene glycol monocaprylate, and combinations thereof.

Perferably the antimicrobial lipid component is recognized as a food additive by the United States Food and Drug Administration and is used in an amount not exceeding the amount permitted by the FDA.

Illustrative examples of tartar control agents that can be used herein include an alpha-hydroxy acid, a beta-hydroxy acid, and a chelating agent in a matrix of edible proteinaceous material that provides long lived chewing and mechanic cleaning of tooth surfaces.

In some embodiments, the pet chew article will further comprise an effective amount of tartar control components. Illustrative examples of tartar control agents that can e used herein include an alpha-hydroxy acid, a beta-hydroxy acid, and a chelating agent in a matrix of edible proteinaceous material that provides long lived chewing and mechanic cleaning of tooth surfaces. In certain embodiments, the tartar control component preferably includes a chelator. In certain embodiments, the tartar control component preferably includes hexametaphosphoric acid and its salts and pyrophosphoric acid and its salts. The tartar control component also serves to enhance the antimicrobial activity especially against Gram negative bacteria, such as E. coli and Pseudomonas sp.

In some embodiments, the article of the invention will further comprise optional additives for the purpose of providing some desired performance. For example, chew articles of the invention may further comprise additives for imparting desired scent, odor, and flavors, color, etc.

Articles of the invention may contain additional compatible pharmaceutically active materials for combination therapy (such as supplementary antimicrobials, anti-caries agents, anti-parasitic agents, vitamins, antipruritics, astringents, local anesthetics, steroids, non-steroidal anti-inflammatory agents, or other anti-inflammatory agents), or may contain materials useful in physically formulating various dosage forms of the present invention, such as excipients, dyes, perfumes, lubricants, mold release agents, fibers, plasticizers, stabilizers, flavoring agents, preservatives, or antioxidants.

Articles of the invention may further include fibers such as cellulose fiber, wheat bran, innulin, wood fiber, sisal fiber, and mixtures thereof. The fibers may be added in amounts up to 60% by weight of the composition. The fibers are added as filler and to adjust the texture of the chew article where the ease of chewing the gluten matrix is generally increased by the addition of fiber. The fiber content is preferably from 1% to 20% of the composition by total weight. Starches that resist digestion can also be added as filler materials. Highly cross-linked wheat starch manufactured, for example, by processes disclosed in U.S. Pat. No. 5,855,946 and U.S. Pat. No. 6,299,907 (both Seib et al.) is representative. Such fillers reduce the overall caloric content of the chew article, making them less fattening for the animal.

It will also be appreciated that additional antiseptics, disinfectants, or antibiotics may be included and are contemplated. These include, for example, addition of metals or metal ions such as silver, copper, zinc; chlorhexidine and its various salts such as chlorhexidine digluconate; polyhexamethylenebiguanide, parachlorometaxylenol, triclosan, and antimicrobial quaternary amines including polymeric quaternary amines.

In some embodiments, chews of the invention will further comprise therapeutic enzymes, which herein means that they cause (e.g., by catalysis) the decomposition of harmful carbohydrates, proteins, lipids, and/or bacterial substrates in the mouth of a subject (e.g., in the oral plaque and saliva). A preferred group of enzymes generate bactericidal products (e.g., H₂O₂). A second preferred group of enzymes are capable of hydrolyzing and solubilizing the carbohydrate biofilm that plaque-causing bacteria generate.

Optionally, one or more enzyme substrates can be incorporated in articles of the present invention to enhance the therapeutic function of the enzymes. For example, for systems requiring water for activation, an enzyme substrate could be present in the composition with the enzymes if the system is anhydrous until use, thereby keeping the enzymes and substrate from interacting.

Method

In one embodiment, the present invention provides a method of removing plaque from an animal's teeth comprising providing the animal with a pet chew article as described herein and chewing the article.

As the animal chews on the article, the mechanical action of rubbing its teeth against the surface of the chew article with the exterior surface having the geometry defined herein, results in plaque and calculus being removed from the teeth. If the chew article comprises other materials such as antimicrobial agents or breath freshening agents, those are activated. As the animal chews, pieces of the chew article are broken off and swallowed by the animal. In accordance with the present invention, none of those pieces is shaped such that no interior point of the piece is more than a selected distance from the portion of the exterior surface nearest to that point. As a result, the pieces which are swallowed pass readily through the animal's digestive tract.

The above summary of the present invention is not intended to describe each disclosed embodiment or every implementation of the present invention. In several places throughout the application, guidance is provided through lists of examples, which examples can be used in various combinations. In each instance, the recited list serves only as a representative group and should not be interpreted as an exclusive list.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A pet chew article comprising a consumable body, said body having a shape defined by an exterior surface and comprising a main portion and at least one protrusion extending therefrom with the proviso that the shape of said body is such that no point in the interior of said body is more than about 0.20 inch (5.1 millimeters) from the portion of said exterior surface nearest to said point.

2. The article of claim 1 with the proviso that the shape of said body is such that no point in the interior of said body is more than about 0.1875 inch (4.8 millimeters) from the portion of said exterior surface nearest to said point.

3. The article of claim 1 with the proviso that the shape of said body is such that no point in the interior of said body is more than about 0.15 inch (3.8 millimeters) from the portion of said exterior surface nearest to said point.

4. The article of claim 1 with the proviso that the shape of said body is such that no point in the interior of said body is more than about 0.125 inch (3.2 millimeters) from the portion of said exterior surface nearest to said point.

5. The article of claim 1 wherein said body comprises an edible chew base selected from the group consisting of wheat gluten, gelatinized starch, and combinations thereof.

6. The article of claim 1 wherein said body comprises: (a) edible chew base, (b) an effective amount of antimicrobial lipid, and (c) at least one of (1) an effective amount of tartar control agent or (2) an effective amount of a long chain fatty acid.

7. The pet chew article of claim 1 wherein said article is at least about 1 inch (25 millimeters) in both the X and Y axes and less than about 0.4 inch (10 millimeters) in the Z axis.

8. The pet chew article of claim 7 wherein said article is at least about 2 inches (50 millimeters) in at least one of the X and Y axes.

9. The pet chew article of claim 7 wherein said article is less than about 0.375 inch (9.5 millimeters) in the 2 axis.

10. The pet chew article of claim 1 wherein said article is at least about 3 inches (75 millimeters) in at least one dimension.

11. A process for the prevention of tartar accumulation on the teeth of a dog or cat, comprising chewing or eating by the dog or cat of an article of claim 1.
